(19) United States
(12) Patent Application Publication
(10) Pub. No.: US 2003/0171762 A1
(43) Pub. Date: Sep. 11, 2003

(54) Instrument dispenser

(76) Inventors: Mark J. Forchette, Colleyville, TX (US); Dyson W. Hickingbotham, Stouchsburg, PA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/407,792

(22) Filed: Apr. 4, 2003

Related U.S. Application Data
(63) Continuation-in-part of application No. 10/162,154, filed on Jun. 4, 2002.
Application No. 10/162,154 is a continuation of application No. 09/641,066, filed on Aug. 17, 2000, now Pat. No. 6,488,695.

(51) Int. Cl. A61B 17/10

(52) U.S. Cl. 606/139

(57) ABSTRACT

An instrument dispenser having an actuation handle made from springy material having a memory. Squeezing the handle causes the dispenser to elongate, thereby causing movement in the distal end of the dispenser tip. The tip has a ratcheting mechanism so that the movement is in the forward direction only. The tip further contains a sclerotomy lance with a shaft slightly larger in diameter than an aperture in the wound clamp. A plurality of wound clamps may be held on the tip about the lance so that the shaft of the lance spreads the halves of the wound clamps apart slightly. The wound clamps are pushed forward by the ratcheting movement of the tip and deposited at the surgical site. The dispenser of the present invention may be held and actuated in any position by a single hand.

This application is a continuation-in-part application of U.S. patent application Ser. No. 10/162,154, filed Jun. 4, 2002, currently co-pending, which is a continuation of U.S. patent application Ser. No. 09/641,066, filed Aug. 17, 2000, now U.S. Pat. No. 6,488,695 B1.

### BACKGROUND OF THE INVENTION

This invention relates generally to the field of ophthalmic surgery and, more particularly, to posterior segment ophthalmic surgery.

During ophthalmic microsurgery, it is often necessary to dissect, cut, delaminate or otherwise manipulate delicate tissues within the eye. Microsurgical tools, such as microscissors, micro forceps and other devices generally are used for such manipulations. Many of these devices require some sort of actuation (i.e., the blades of a scissors must be rotated across each other in order to cut, the grasping tips of a forceps must be bought together in order to grasp, etc.). These devices generally are inserted through one or more surgical incisions in the conjunctiva and sclera, a sclerotomy, and different tools may be used during different parts of the surgical procedure. The repeated insertion and removal of these instruments can allow vitreous humor and fluids to escape the eye out through the sclerotomy, increasing the potential for softening of the globe, bleeding, traction on the retina and/or introduction of bacteria into the eye as well as increased healing time. To reduce the trauma associated with the insertion and removal of these surgical devices, various cannulas or trocars are used. One new device, a wound clamp disclosed in U.S. patent application Ser. No. 10/337,532, filed Jan. 7, 2003, the entire contents of which being incorporated herein by reference, discloses a wound clamp having two substantially identical halves. The clamp halves are biased together using a spring or elastomeric band. Prongs or needles integrally formed with the halves cause the clamp to be engaged firmly on the conjunctival and scleral tissue about the wound. The biasing of the clamp halves toward each other provide for a tight sealing of the wound, but allows entry and removal of microsurgical tools.

The installation of the wound clamp described above can be difficult, because the two halves of the clamp need to be spread apart prior to contacting the tissue, then released so that the prongs bite into the tissue and hold the clamp firmly. In addition, the wound clamp aperture must be aligned with the sclerotomy to work well.

Therefore, a need continues to exist for a simple, inexpensive dispenser for an ophthalmic wound clamp.

### BRIEF SUMMARY OF THE INVENTION

The present invention improves upon the prior art by providing an instrument dispenser having an actuation handle made from springy material having a memory. Squeezing the handle causes the dispenser to elongate, thereby causing movement in the distal end of the dispenser tip. The tip has a ratcheting mechanism so that the movement is in the forward direction only. The tip further contains a sclerotomy lance with a shaft slightly larger in diameter than an aperture in the wound clamp. A plurality of wound clamps may be held on the tip about the lance so that the shaft of the lance spreads the halves of the wound clamps apart slightly. The wound clamps are pushed forward by the ratcheting movement of the tip and deposited at the surgical site. The dispenser of the present invention may be held and actuated in any position by a single hand.

Accordingly, one objective of the present invention is to provide a simple dispenser actuation handle.

Another objective of the present invention is to provide an inexpensive dispenser actuation handle.

Still another objective of the present invention is to provide a dispenser handle that may be held and actuated in any position by a single hand.

Yet another objective of the present invention is to provide a dispenser handle that may be used to install spring-loaded wound clamps on scleral tissue.

Another objective of the present invention is to provide a dispenser handle that may be used to create a sclerotomy incision and align and install a spring-loaded wound clamp or trocar.

These and other advantages and objectives of the present invention will become apparent from the detailed description and claims that follow.

### BRIEF DESCRIPTION OF THE DRAWING

FIG. 1 is an exploded perspective view of dispenser of the present invention.

FIG. 2 is a perspective view of the dispenser of the present invention.

### DETAILED DESCRIPTION OF THE INVENTION

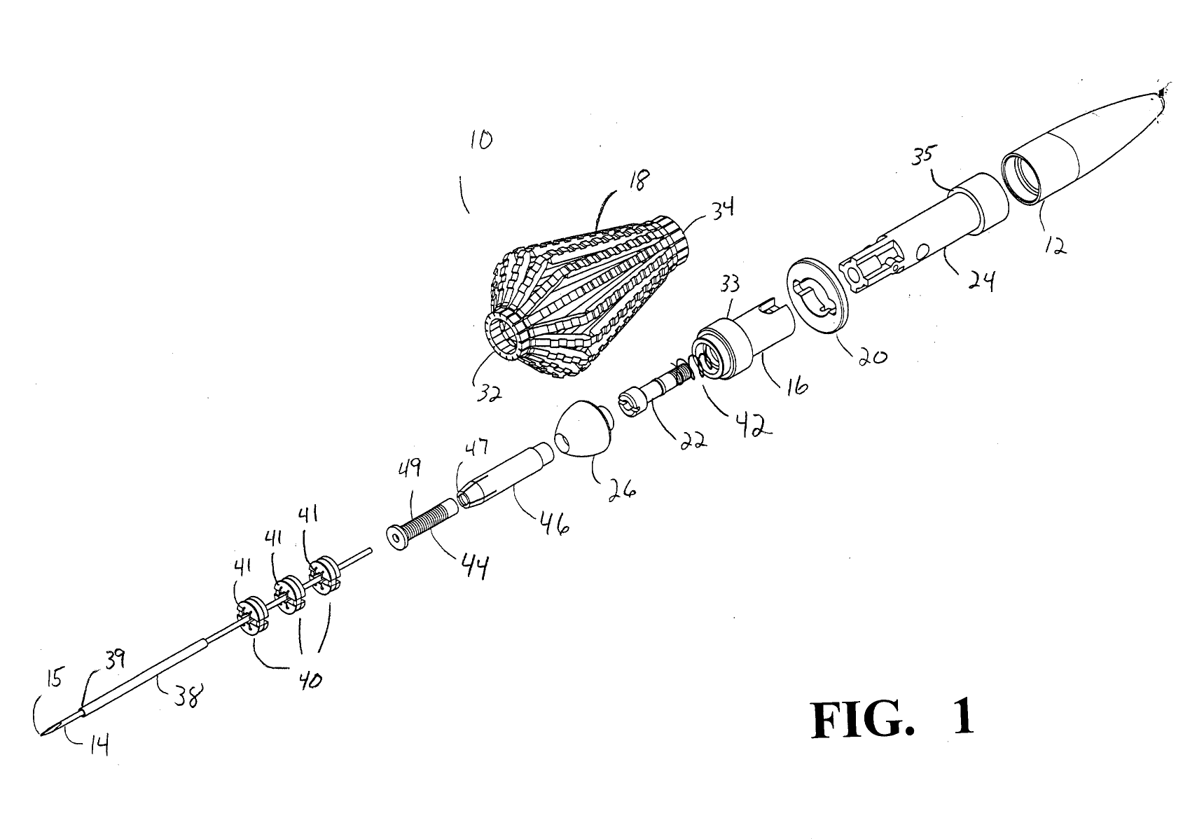

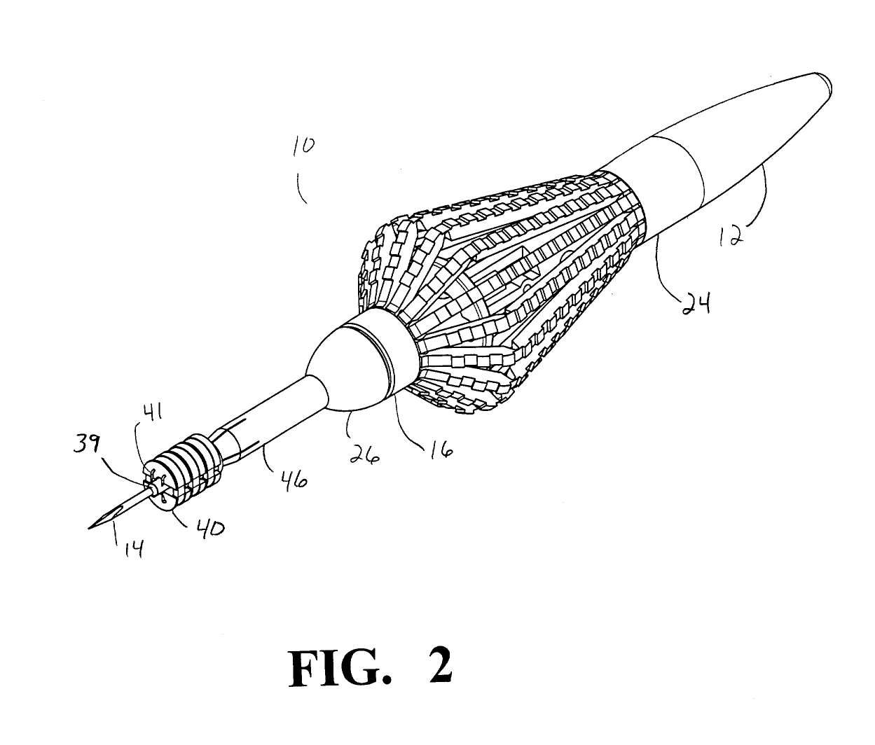

As seen in FIGS. 1 and 2, dispenser 10 generally consists of end cap 12, lance 14, dispenser actuation sleeve 16 and dispenser actuation handle 18. End cap 12 may be made of any suitable material such as injection molded or machined thermoplastic or metal and may be textured or knurled to improve gripping of end cap 12. Lance 14 may be any suitable type and size of knife and will generally be made from stainless steel or titanium, but other materials may also be used. Distal tip 15 of lance 14 is suitably sized and sharpened to penetrate the targeted tissue. Sleeve 16 may be made of any suitable material such as injection molded or machined thermoplastic or metal. Actuation handle 18 may be made from any suitable springy material having a memory, such as titanium, stainless steel or suitable thermoplastic.

Dispenser 10 is assembled by placing spacer 20 within handle 18 and then placing adjustment screw 22 first through actuation sleeve 16 through handle 18 and screwing screw 22 into end sleeve 24 so that distal end 32 of handle 18 is abuts against rim 33 on sleeve 16 and proximal end 34 of handle 18 abuts against rim 35 on end sleeve 24. Sleeve 16 is not attached to screw 22, but reciprocates on screw 22. Screw 22 allows for adjustment of the length of the stroke of sleeve 16 during actuation, as will be described below. When so assembled, spacer 20, the proximal portion of sleeve 16 behind rim 33 and the distal portion of end sleeve 24, located distally of rim 35 are surrounded by handle 18. Lance 14 is inserted through and fixed (e.g., welded) to tubular member 38. Lance 14 is then inserted through wound clamps 40, ridged sleeve 44, ratcheting sleeve 46, nose cone 26, adjustment screw 22, return spring 42, sleeve 16 and spacer 20 and secured to sleeve by any suitable method such as an adhesive or set screws (not shown). When so assembled, ridged sleeve 44 is telescopically received within ratcheting sleeve 46. End cap 12 is pressed into end sleeve 24. Tubular member 38 is sized slightly larger than the aperture on wound clamps 40 so as to slightly force apart the halves of wound clamps 40. Distal end 39 of tubular member 38 is slightly tapered or ramped toward distal end 15 of lance 14. Such a construction allows for easier removal of clamps 40 from tubular member 38 as will be described below.

In use, squeezing of actuation handle 18 forces distal end 32 of actuation handle 18 forward, because rearward movement of proximal end 34 of actuation handle 18 is prevented by end sleeve 24. The forward movement of distal end 32 of actuation handle 18 is transferred to sleeve 16 through rim 33, causing sleeve 16 to slide distally (forward), pushing nosecose 26, ratcheting sleeve 46, ridged sleeve 44 and clamps 40 forward. When handle 18 is released, the spring nature of handle 18 causes handle 18 return to its original shorter, but larger in diameter shape. Sleeve 16, nosecone 26 and ratcheting sleeve 46 also are pulled backward toward end sleeve 24. Lance 14 remains in place, and does not move with the squeezing of handle 18. Ridged sleeve 44, and clamps 40 are pushed forward by ratcheting sleeve 46, but remain in place upon retraction of ratcheting sleeve 46 because of a tight frictional fit between ridged sleeve 44 and tubular member 38. As ratcheting sleeve 46 retracts from ridged sleeved 44, distal edge 47 of ratcheting sleeve 46 engage proximal ridges 49 on ridged sleeve 44 that are closer to nosecone 26. Subsequent squeezing cycles of handle 18 cause ridged sleeve 44 and clamps 40 to be forced down sleeve 38 to the surgical site until prongs 41 engage the tissue at the surgical site. The amount of movement of sleeve 16 can be controlled easily by varying the outer diameter of actuation handle 18 in its relaxed stated, with larger diameters causing greater longitudinal movement. Spacer 20 prevents handle 18 from being over-actuated.

In surgery, dispenser is placed so that distal tip 15 of lance 14 penetrates a surgical site, such as the sclera. Handle 18 is repeatedly squeezed and allowed to relax so that clamps 40 are forced down lance 14 to the surgical site until prongs 41 engage the tissue at the surgical site. Dispenser 10 is then removed from the surgical site and the halves of clamps 40 close together and secure themselves to the sclera because the halves are no longer being forced apart by tubular member 38.

This description is given for purposes of illustration and explanation. It will be apparent to those skilled in the relevant art that changes and modifications may be made to the invention described above without departing from its scope or spirit.

We claim:

1. An instrument dispenser, comprising:
a) a compressible actuation handle having a proximal end and a distal end;
b) an actuation sleeve on the distal end of the actuation handle;
c) a ratcheting sleeve connected to the actuation sleeve;
d) a ridged sleeve telescopically received within the ratcheting sleeve; and
d) a lance telescopically received through the ridged sleeve, the ratcheting sleeve and the actuation sleeve, the ridged sleeve being fixed to the lance so that compression of the actuation handle causes elongation of the actuation handle and resulting reciprocal movement of the actuation sleeve and the ratcheting sleeve along the lance.

2. The dispenser of claim 1 further comprising an end sleeve fixed to the proximal end of the actuation handle and an adjusting screw received in the end sleeve.

3. The dispenser of claim 1 further comprising a wound clamp frictionally received on the lance.

4. The dispenser of claim 1 further comprising a tubular member telescopically received over the lance.

5. The dispenser of claim 4 wherein the tubular member is tapered toward a distal end of the lance.

6. The dispenser of claim 4 further wherein the tubular member is sized so as to spread apart slightly the halves of a wound clamp inserted over the tubular member.

7. An instrument dispenser, comprising:
a) an actuation handle having a first diameter and a first length in its relaxed state and a second diameter and a second length in its compressed state, the first diameter being larger than the second diameter and the second length being longer than the first length;
b) an actuation sleeve fixed to the distal end of the actuation handle;
c) an end sleeve fixed to the proximal end of the actuation sleeve;
d) a ratcheting sleeve received on the distal end of the actuation sleeve;
e) a ridged sleeve telescopically received within the ratcheting sleeve; and
d) a lance telescopically received through the ridged sleeve, the ratcheting sleeve and the actuation sleeve, the ridged sleeve being fixed to the lance so that compression of the actuation handle causes reciprocal movement of the actuation sleeve and the ratcheting sleeve along the lance.

8. The dispenser of claim 7 further comprising an end sleeve fixed to the proximal end of the actuation handle and an adjusting screw received in the end sleeve.

9. The dispenser of claim 7 further comprising a wound clamp frictionally received on the lance.

10. The dispenser of claim 7 further comprising a tubular member telescopically received over the lance.

11. The dispenser of claim 10 wherein the tubular member is tapered toward a distal end of the lance.

12. The dispenser of claim 10 further wherein the tubular member is sized so as to spread apart slightly the halves of a wound clamp inserted over the tubular member.
